(19) United States
(12) Patent Application Publication
(10) Pub. No.: US 2018/0129033 A1
(43) Pub. Date: May 10, 2018

(54) OPTICAL-SCANNING OBSERVATION DEVICE

(71) Applicant: OLYMPUS CORPORATION, Tokyo (JP)

(72) Inventor: Yuhei TAKATA, Tokyo (JP)

(73) Assignee: OLYMPUS CORPORATION, Tokyo (JP)

(21) Appl. No.: 15/862,835

(22) Filed: Jan. 5, 2018

Related U.S. Application Data
(63) Continuation of application No. PCT/JP2015/071035, filed on Jul. 23, 2015.

(51) Int. Cl. G02B 23/26; A61B 1/00; G02B 23/24; G02B 26/10; A61B 1/07

(52) U.S. Cl. CPC G02B 23/26; A61B 1/00172; G02B 23/2469; G02B 23/2476; G02B 26/103; H04N 2005/2255; A61B 1/07; A61B 1/00057; A61B 1/00006; G01N 2201/0697; A61B 1/00165

(57) ABSTRACT

An optical-scanning observation device including: a laser output unit that outputs a laser beam; a light scanner that radiates the laser beam on an object while scanning; a light detector that detects reflected light of the laser beam; and a controller that sets a detection delay time on the basis of a propagation delay time from when the laser beam is output from the laser output unit to when the reflected light is detected by the light detector and that controls the laser output unit and the light detector such that the light detector detects the reflected light after the detection delay time has elapsed since the laser beam is output from the laser output unit.

### CROSS-REFERENCE TO RELATED APPLICATIONS

This is a Continuation Application of International Application No. PCT/JP2015/071035 filed on Jul. 23, 2015. The content of International Application No. PCT/JP2015/071035 is hereby incorporated herein by reference in its entirety.

### TECHNICAL FIELD

The present invention relates to optical-scanning observation devices.

### BACKGROUND ART

A known related-art optical-scanning observation device acquires an image while scanning a laser beam over an object (for example, see PTL 1). In this optical-scanning observation device, the laser beam output from a laser light source is irradiated on the object through an illumination optical fiber, and reflected light of the laser beam reflected by the object is detected by a photodetector through a light-receiving optical fiber. Then, the intensity of the reflected light detected by the photodetector is associated with the scanning position of the laser beam, whereby an image of the object is generated.

### CITATION LIST

### Patent Literature

{PTL 1} The Publication of Japanese Patent No. 5235651

### SUMMARY OF INVENTION

The present invention provides an optical-scanning observation device including: a laser output unit that outputs a laser beam; a light scanner that radiates the laser beam output from the laser output unit toward an object while scanning the laser beam in a direction intersecting an optical axis of the laser beam; a light detector that detects reflected light of the laser beam reflected by the object; and a controller that controls the laser output unit and the light detector such that the light detector detects the reflected light after a predetermined detection delay time has elapsed since the laser beam is output from the laser output unit. The controller sets the detection delay time on the basis of a propagation delay time from when the laser beam is output from the laser output unit to when the reflected light of the laser beam reaches the light detector, the optical-scanning observation device further includes: an illumination optical fiber that guides the laser beam supplied from the laser output unit and emits the laser beam toward the object; and a light-receiving optical fiber that receives the reflected light from the object and guides the received reflected light to the light detector, wherein the controller sets the detection delay time on the basis of a time for the laser beam to propagate thorough the illumination optical fiber and a time for the reflected light to propagate through the light-receiving optical fiber.

Another aspect of the present invention provides an optical-scanning observation device including: a laser output unit that outputs a laser beam; a light scanner that radiates the laser beam output from the laser output unit toward an object while scanning the laser beam in a direction intersecting an optical axis of the laser beam; a light detector that detects reflected light of the laser beam reflected by the object; and a controller that controls the laser output unit and the light detector such that the light detector detects the reflected light after a predetermined detection delay time has elapsed since the laser beam is output from the laser output unit, wherein the controller sets the detection delay time on the basis of a propagation delay time from when the laser beam is output from the laser output unit to when the reflected light of the laser beam reaches the light detector, wherein the controller performs a propagation-delay-time measuring operation in which the propagation delay time is measured, and sets a time equal to the propagation delay time measured in the propagation-delay-time measuring operation as the detection delay time.

### BRIEF DESCRIPTION OF DRAWINGS

FIG. 1 is a diagram showing the overall configuration of an optical-scanning observation device according to an embodiment of the present invention.

FIG. 2 is a flowchart showing a propagation-delay-time measuring operation performed by the optical-scanning observation device in FIG. 1.

FIG. 3 is a flowchart showing a method of controlling a laser output unit and a light detector with a main controller.

FIG. 4 shows the timing at which a laser beam is output from the laser output unit, the timing at which the reflected light reaches a photodetector, and the timing at which the light detector detects the reflected light.

FIG. 5 shows a measuring apparatus that is used to measure the laser-beam and reflected-light propagation times in a housing of the optical-scanning observation device in FIG. 1.

FIG. 6 is a flowchart showing a modification of the propagation-delay-time measuring operation performed by the optical-scanning observation device in FIG. 1.

FIG. 7 shows the timing at which a laser beam is output from the laser output unit, the timing at which the reflected light reaches a photodetector, and the timing at which the light detector detects the laser beam in the propagation-delay-time measuring operation in FIG. 6.

FIG. 8 is a diagram showing the overall configuration of a modification of the optical-scanning observation device according to FIG. 1.

### DESCRIPTION OF EMBODIMENTS

An optical-scanning observation device 1 according to an embodiment of the present invention will be described below with reference to the drawings.

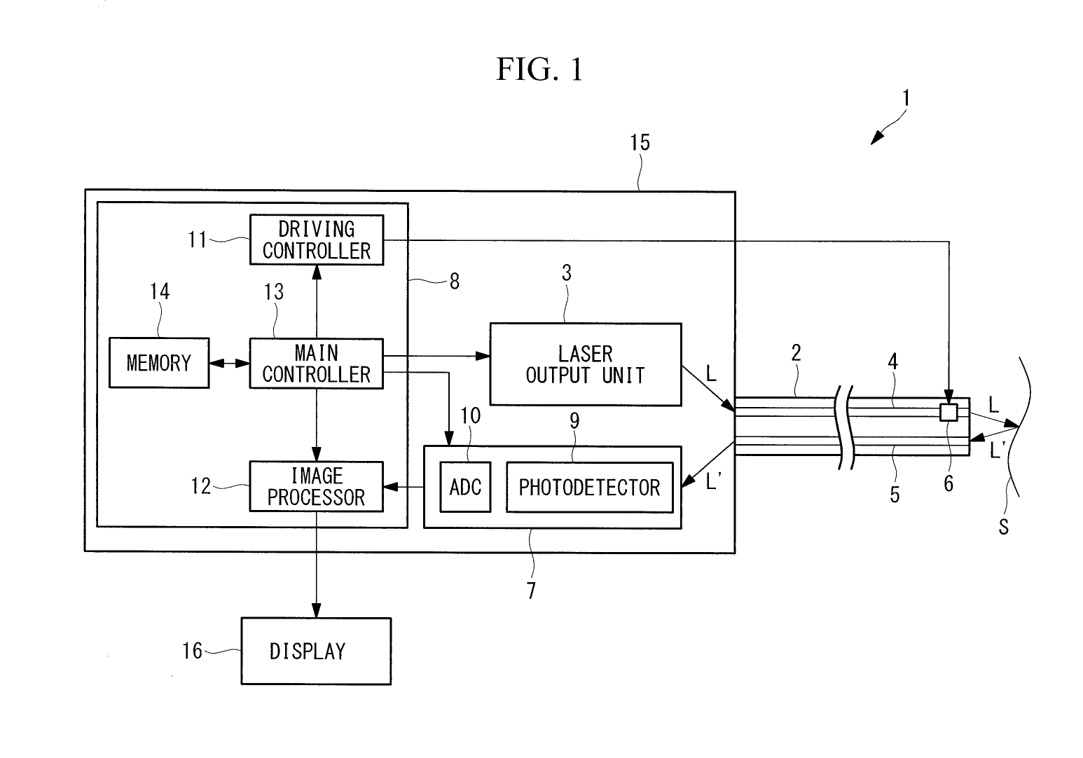

As shown in FIG. 1, the optical-scanning observation device 1 according to this embodiment is an endoscope apparatus having an elongated insertion part 2 that can be inserted into the body. The optical-scanning observation device 1 includes: a laser output unit 3 that outputs a laser beam L; an illumination optical fiber 4 and a light-receiving optical fiber 5 provided inside the insertion part 2; a light scanner 6 that scans the laser beam L emitted from the distal end of the illumination optical fiber 4; a light detector 7 that detects reflected light L′ of the laser beam L reflected by an object S; and a control device 8 that controls the laser output unit 3, the light scanner 6, and the light detector 7.

The laser output unit 3 is provided inside a housing 15 connected to the proximal end of the insertion part 2. The laser output unit 3 includes three laser light sources (not shown), which generate red (R), green (G), and blue (B) laser beams L, and sequentially and repeatedly outputs the pulsed R, G, and B laser beams L.

The illumination optical fiber 4 is a single-mode fiber. The illumination optical fiber 4 is disposed inside the insertion part 2, along the longitudinal direction of the insertion part 2, and is connected to the laser output unit 3 at the proximal end thereof. The laser beam L coming from the laser output unit 3 and incident on the proximal-end surface of the illumination optical fiber 4 is guided inside the illumination optical fiber 4, from the proximal end to the distal end thereof, and is emitted from the distal-end surface of the illumination optical fiber 4 toward the front side of the distal end of the insertion part 2.

The light-receiving optical fiber 5 is a multi-mode fiber. The light-receiving optical fiber 5 is disposed parallel to the illumination optical fiber 4, and the distal end surface of the light-receiving optical fiber 5 is disposed at the distal end surface of the insertion part 2. Although FIG. 1 shows only one light-receiving optical fiber 5, a plurality of light-receiving optical fibers 5 may be provided so as to surround the illumination optical fiber 4 in the circumferential direction.

The light scanner 6 is, for example, a piezoelectric actuator including a piezoelectric element and is attached to the distal end of the illumination optical fiber 4. By receiving the supply of a driving voltage from a driving controller 11 (described below), the light scanner 6 vibrates the distal end of the illumination optical fiber 4 along a spiral path. As a result, the laser beam L emitted from the distal end surface of the insertion part 2 is scanned along a spiral scanning path.

The light detector 7 includes a photodetector 9, such as a photodiode or a photomultiplier tube, and an A/D converter 10 and is provided inside the housing 15.

The proximal end of the light-receiving optical fiber 5 is connected to the photodetector 9. The reflected light L′ coming from the object S and incident on the distal end surface of the light-receiving optical fiber 5 is guided through the light-receiving optical fiber 5 from the distal end to the proximal end and is incident on the photodetector 9. The photodetector 9 outputs an electric signal corresponding to the intensity of the reflected light L′ incident thereon. The A/D converter 10 converts the electric signal output from the photodetector 9 into a digital signal and then outputs the generated digital signal to an image processor 12 (described below).

The control device 8 includes the driving controller 11 that controls the light scanner 6, the image processor 12 that generates an image of the object S, a main controller (controller) 13 that performs overall control of the units 3, 6, 7, 11, and 12, and a memory (storage unit) 14.

The driving controller 11 generates a driving voltage and supplies the generated driving voltage to the light scanner 6 according to a control signal from the main controller 13.

The image processor 12 generates an image of the object S by acquiring, from the main controller 13, information about the scanning position of each laser beam L output from the laser output unit 3 and associating the digital signal received from the A/D converter 10 with the scanning position. The generated image is displayed on the display 16 disposed outside the housing 15.

The main controller 13 controls the laser output unit 3 such that it sequentially outputs pulsed R, G, and B laser beams L at predetermined time intervals. Furthermore, the main controller 13 controls the light detector 7 such that it detects the reflected light L′ at the same time intervals as the output intervals of the laser beam L from the laser output unit 3. The output intervals of the laser beam L and the detection intervals of the reflected light L′ are, for example, 0.01 to 1 μs (the output rate and the detection rate are 1 MHz to 100 MHz).

At this time, the main controller 13 controls the timing at which the light detector 7 detects the reflected light L′ with respect to the timing at which the laser output unit 3 outputs the laser beam L such that the light detector 7 detects the reflected light L′ with a delay of the detection delay time after the laser output unit 3 outputs the laser beam L. The detection delay time is set to a time equal to the propagation delay time measured in the propagation-delay-time measuring operation.

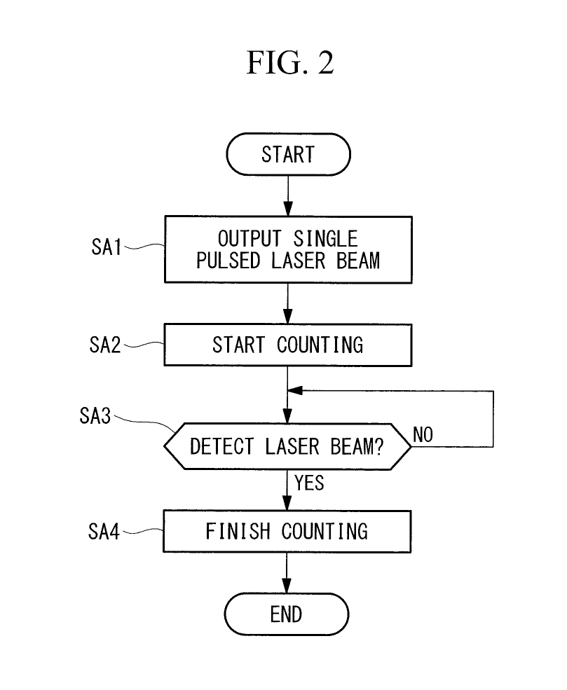

FIG. 2 is a flowchart showing the propagation-delay-time measuring operation.

As shown in FIG. 2, the main controller 13 makes the laser output unit 3 output a single pulsed laser beam L (step SA1) and, at the same time, starts to count time with a timer (not shown) (step SA2). The main controller 13 continues to count time until the light detector 7 detects reflected light L′ (“NO” in step SA3), and when the light detector 7 has detected the reflected light L′ (“YES” in step SA3), the main controller 13 stops counting (step SA4). This way, the propagation delay time from when the laser beam L is output from the laser output unit 3 to when the reflected light L′ of the laser beam L reaches the photodetector 9 is obtained in step SA4.

The measured propagation delay time is stored in the memory 14. Hence, once the propagation delay time is measured, the main controller 13 can use the propagation delay time stored in the memory 14 to set the detection delay time.

Note that, although the observation distance between the distal end of the insertion part 2 and the object S varies, because the observation distance is sufficiently small as compared with the length of the insertion part 2, changes in the propagation delay time due to variations in the observation distance are small enough to be ignored.

The propagation-delay-time measuring operation is performed every time the optical-scanning observation device 1 is started. Alternatively, it is also possible to perform, by using a white chart or the like as the object S, the propagation-delay-time measuring operation before shipment of the optical-scanning observation device 1 and then shipping the optical-scanning observation device 1 in a state in which the measured propagation delay time is stored in the memory 14. This saves a user the effort of measuring the propagation delay time.

This control device 8 is constituted by a computer, and processing performed by the driving controller 11, the image processor 12, and the main controller 13 is executed by an arithmetic processing device installed in the computer. More specifically, control programs for controlling the units 3, 6, 7, 11, and 12 and an image processing program for generating images are stored in the memory 14. The arithmetic processing device realizes the aforementioned processing performed by the driving controller 11, the image processor 12, and the main controller 13 by executing these programs stored in the memory 14.

Next, the effect of the thus-configured optical-scanning observation device 1 will be described.

When the supply of a driving voltage from the driving controller 11 to the light scanner 6 and output of a laser beam L from the laser output unit 3 are started, pulsed R, G, and B laser beams L are sequentially emitted from the distal end of the illumination optical fiber 4, which vibrates in a spiral manner. As a result, the R, G, and B laser beams L are sequentially irradiated, along a spiral scanning path, on the surface of the object S facing the distal end surface of the insertion part 2.

The reflected light L′ of the laser beam L reflected by the surface of the object S is received by the light-receiving optical fiber 5, and the intensity of the reflected light L′ is detected by the light detector 7. In the image processor 12, the intensity of the detected reflected light L′ is associated with the scanning position thereof, and thus, an image of the object S is generated.

In this case, the laser beam L output from the laser output unit 3 goes and returns through the elongated insertion part 2 and is then detected by the light detector 7. When the insertion part 2 has a length of 2 m to 4 m, the distance over which the laser beam L and the reflected light L′ propagate from when it is output from the laser output unit 3 to when it reaches the photodetector 9 is about 4 m to 8 m, and the propagation delay time is on the order of a few nanoseconds.

This magnitude of propagation delay time cannot be ignored in the detection with the light detector 7 at a detection rate of 1 MHz to 100 MHz. Specifically, when the light detector 7 detects the reflected light L′ at the same time as when the laser beam L is output from the laser output unit 3, the detection is performed before the reflected light L′, serving as a detection target, reaches the photodetector 9, and thus, the correct intensity of the reflected light L′ cannot be detected. Furthermore, as a result of the photodetector 9 detecting the reflected light L′ preceding the detection-target reflected light L′, color misalignment or pixel misalignment may occur.

Furthermore, the propagation delay time varies with, besides the length of the insertion part 2 (i.e., the length of the optical fibers 4 and 5), the refractive indices and types of propagation mode of the optical fibers 4 and 5.

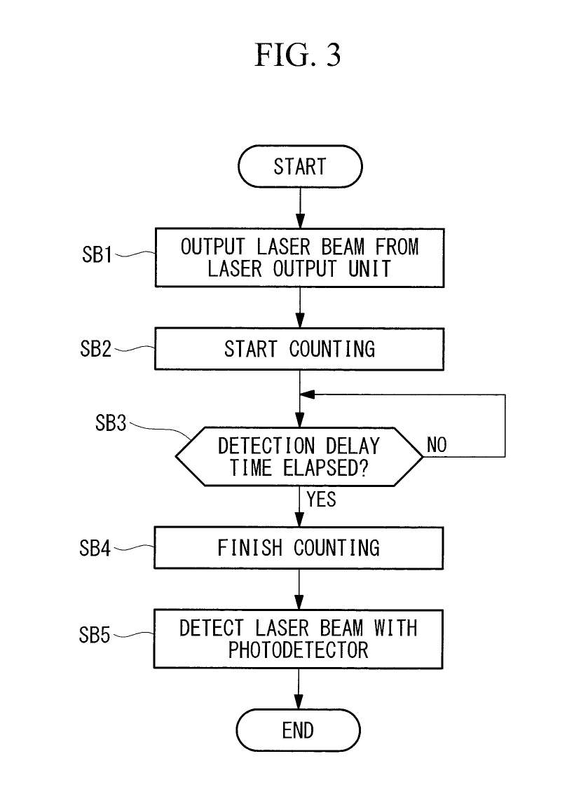

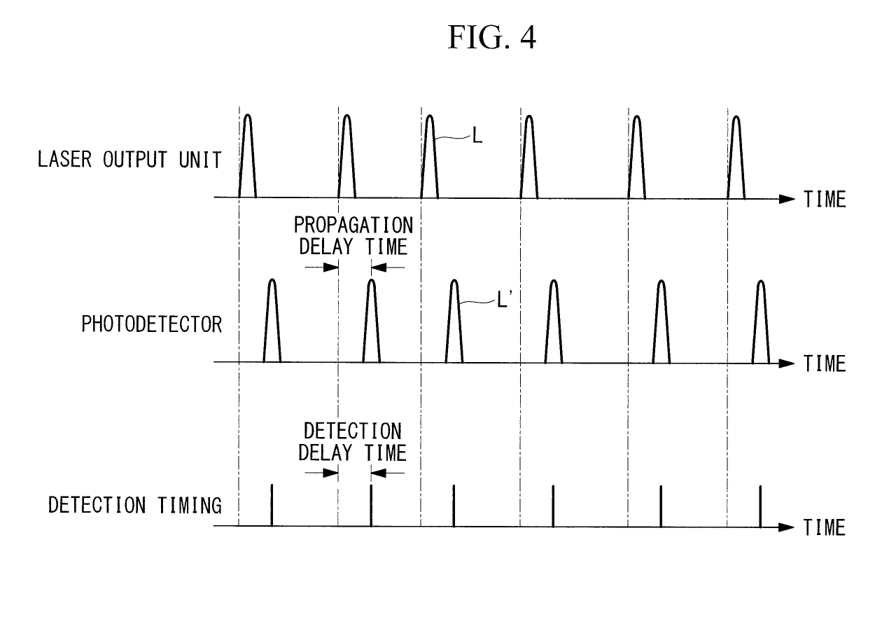

As shown in FIG. 3, in this embodiment, the main controller 13 makes the laser output unit 13 output a laser beam L (step SB1), waits until the detection delay time has elapsed (steps SB2, SB3, and SB4), and makes the light detector 7 to perform detection of reflected light L′ (step SB5). At this time, the detection delay time is set to a time equal to the actually measured propagation delay time. Hence, as shown in FIG. 4, the light detector 7 performs detection at the time at which the reflected light L′ reaches the photodetector 9, and thus, the light detector 7 can detect the accurate intensity of the reflected light L′. Furthermore, the image processor 12 can associate the intensity of the reflected light L′ with the correct scanning position. This leads to an advantage in that it is possible to acquire an accurate image of the object S that is free from a decline in brightness, color misalignment, or pixel misalignment.

In this embodiment, although the propagation delay time required for the laser beam L and the reflected light L′ to propagate through the overall propagation path from the laser output unit 3 to the photodetector 9 is measured, instead of this, the sum of the propagation time required for the laser beam L to propagate through the illumination optical fiber 4 and the propagation time required for the reflected light L′ to propagate through the light-receiving optical fiber 5 may be used as the propagation delay time.

The propagation delay time is mainly caused by the laser beam L and the reflected light L′ propagating through the long optical fibers 4 and 5 having high refractive indices. Hence, the sum of the propagation times through the respective optical fibers 4 and 5 is substantially equal to the actual propagation delay time. The propagation times through the optical fibers 4 and 5 may be either experimentally measured or theoretically calculated.

When the sum of the propagation times through the optical fibers 4 and 5 is used as the propagation delay time, the propagation time of the laser beam L in the housing 15 may be additionally taken into consideration.

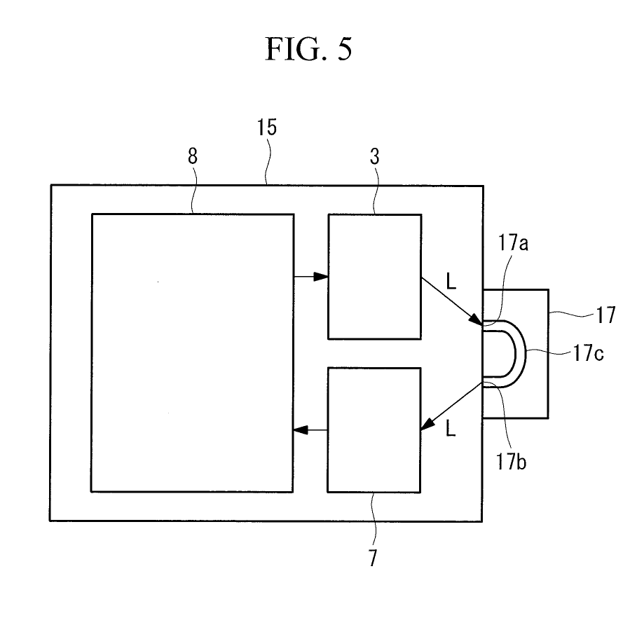

FIG. 5 shows a measuring apparatus 17 that is used to measure the propagation time of the laser beam L in the housing 15. The measuring apparatus 17 is tightly attachable to the housing 15 and has an incident port 17a from which the laser beam L output from the housing 15 enters, an emitting port 17b from which the laser beam L exits into the housing 15, and a waveguide path 17c along which the laser beam L entering from the incident port 17a is guided to the emitting port 17b. The optical path length of the laser beam L in the waveguide path 17c is constant, and the propagation time of the laser beam L in the waveguide path 17c is known. Note that FIG. 5 shows a simplified configuration of the interior of the housing 15.

By performing the propagation-delay-time measuring operation in FIG. 2 in a state in which the measuring apparatus 17 is attached to the housing 15 and by subtracting the propagation time of the laser beam L in the waveguide path 17c from the measured propagation delay time, the propagation time of the laser beam L in the housing 15 is calculated. Then, the sum of the propagation time in the housing 15 and the propagation time through the two optical fibers 4 and 5 is set as the propagation delay time, that is, the detection delay time. In this way, the detection delay time can be set more accurately.

Furthermore, in this embodiment, although the propagation delay time is measured by using a single pulsed laser beam L, instead, the propagation delay time may be measured by using a plurality of pulsed laser beams L.

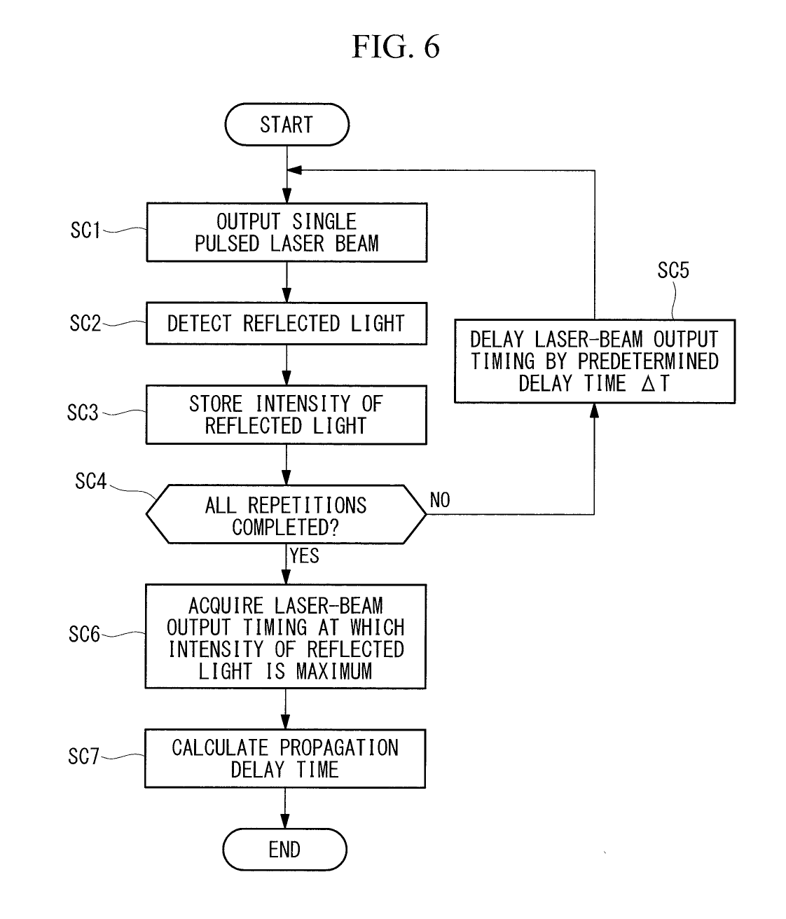

FIG. 6 is a flowchart showing a propagation-delay-time measuring operation in which a plurality of pulsed laser beams L are used. In this measuring operation, the main controller 13 makes the laser output unit 3 output pulsed laser beams L at time intervals (step SC1), makes the photodetector 9 detect reflected light L′ at a certain time period (detection period) (step SC2), and makes the memory 14 store the detected intensities of the reflected light L′ (step SC3).

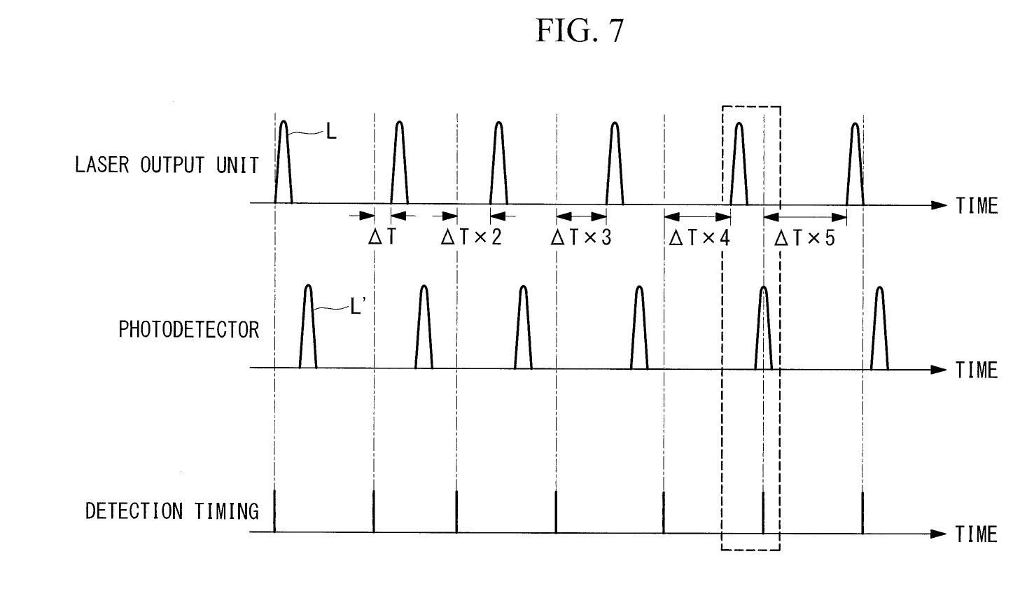

At this time, as shown in FIG. 7, the main controller 13 controls the output timing of the laser beams L from the laser output unit 3 such that the first laser beam L is output at the same time as the timing of detection with the photodetector 9, and the second and subsequent laser beams L are delayed by a predetermined delay time ΔT with respect to the timing of detection with the photodetector 9 (step SC5). As a result, the phase of the output timing of the laser beams L from the laser output unit 3 in the detection period of the reflected light L′ with the photodetector 9 changes. The intensities of the reflected light L′ are associated with delay times (ΔT, ΔT×2, ΔT×3, . . . ) of the output timing of the laser beam L with respect to the timing of detection with the photodetector 9 and are stored in the memory 14.

The main controller 13 repeats steps SC1 to SC5 until the delay time of the output timing of the laser beam L reaches or exceeds the time of one detection period with the photodetector 9 (YES in step SC4). Next, the main controller 13 extracts, from the intensities of the reflected light L′ stored in the memory 14, the delay time that is associated with the maximum intensity (step SC6) and calculates the propagation delay time on the basis of the extracted delay time (step SC7).

Also in this way, the propagation delay time can be accurately measured.

Furthermore, in this embodiment, the propagation-delay-time measuring operation may be performed in parallel with image acquisition.

In this case, the main controller 13 performs the propagation-delay-time measuring operation by using a laser beam L in a part of the period of time for which the laser beam L is scanned by the scanner 6 over the object S. Preferably, the part of the period of time is the period of time for which the laser beam L is scanned over the central part of the spiral scanning path.

The irradiation density of the laser beam L is high in the central part of the scanning path, and the irradiation density of the laser beam L is low at the peripheral part of the scanning path. Hence, a portion of the laser beam L irradiated on the central part of the scanning path is not used for image generation. By measuring the propagation delay time by using the laser beam L that is not used for image generation in this manner, it is possible to perform image acquisition and the propagation-delay-time measuring operation in parallel.

Furthermore, this embodiment may be configured such that the insertion part 2 is attachable to and detachable from the housing 15 and such that an insertion part 2 to be used can be selected from a plurality of types of insertion parts and changed.

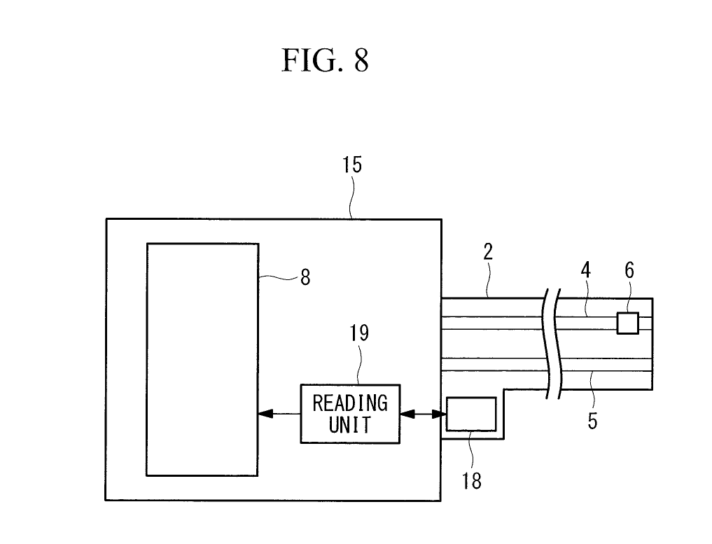

In that case, as shown in FIG. 8, each insertion part 2 is provided with a memory 18, which stores the propagation delay time measured by using the insertion part 2 in which the memory 18 is provided. The housing 15 is provided with a reading unit 19 that reads propagation-delay-time information from the memory 18 of the insertion part 2 that is connected to the housing 15. The main controller 13 sets a detection delay time on the basis of the propagation-delay-time information received from the reading unit 19. Note that, in FIG. 8, illustration of some configurations in the housing 15 is omitted.

With this configuration, even when insertion parts 2 having different lengths or having optical fibers 4 and 5 with different properties are used while being changed, it is possible to set the optimum detection delay time for the insertion part 2 to be used.

From the above-described embodiments, the following aspects of the present invention are derived.

One aspect of the present invention is an optical-scanning observation device including: a laser output unit that outputs a laser beam; a light scanner that radiates the laser beam output from the laser output unit toward an object while scanning the laser beam in a direction intersecting an optical axis of the laser beam; a light detector that detects reflected light of the laser beam reflected by the object; and a controller that controls the laser output unit and the light detector such that the light detector detects the reflected light after a predetermined detection delay time has elapsed since the laser beam is output from the laser output unit. The controller sets the detection delay time on the basis of a propagation delay time from when the laser beam is output from the laser output unit to when the reflected light of the laser beam reaches the light detector.

According to this aspect, the light scanner scans the laser beam output from the laser output unit over the object, and the light detector detects the reflected light of the laser beam coming from the object. As a result, by associating the intensity of the reflected light detected by the light detector with the position thereof on the scanning path, it is possible to generate an image of the object.

In this case, there is a propagation delay time from when the laser beam is output from the laser output unit to when the laser beam, which is now reflected light, is detected by the light detector, according to the optical path lengths of the laser beam and reflected light. On the basis of the propagation delay time, the controller delays the detection of the reflected light with the light detector with respect to the output of the laser beam from the laser output unit. As a result, it is possible to accurately detect, with the light detector, the reflected light at the timing at which the reflected light reaches the light detector, and thus, to acquire an accurate image of the object.

In the above-described aspect, the optical-scanning observation device may further include: an illumination optical fiber that guides the laser beam supplied from the laser output unit and emits the laser beam to the object; and a light-receiving optical fiber that receives the reflected light from the object and guides the received reflected light to the light detector. The controller may set the detection delay time on the basis of a time for the laser beam to propagate thorough the illumination optical fiber and a time for the reflected light to propagate through the light-receiving optical fiber.

In a configuration in which a laser beam and reflected light are guided over a long distance by using optical fibers, laser-beam and reflected-light propagation times through the optical fibers make up most of the propagation delay time. In other words, the sum of the laser-beam propagation time through the illumination optical fiber and the reflected-light propagation time through the light-receiving optical fiber substantially equals the propagation delay time. Hence, it is possible to set an appropriate detection delay time on the basis of the laser-beam and reflected-light propagation times through the optical fibers.

In the above-described aspect, the controller may perform a propagation-delay-time measuring operation in which the propagation delay time is measured, and may set a time equal to the propagation delay time measured in the propagation-delay-time measuring operation as the detection delay time.

With this configuration, by setting the actually measured propagation delay time as the detection delay time, it is possible to more accurately match the time at which the reflected light reaches the light detector and the time at which the light detector detects the reflected light.

In the above-described aspect, the controller may perform the propagation-delay-time measuring operation by using a laser beam in a part of a period of time for which the light scanner performs scanning.

With this configuration, it is possible to perform the propagation-delay-time measuring operation in parallel with image acquisition.

In the above-described aspect, the optical-scanning observation device may further include a memory that stores the propagation delay time. The controller may set the detection delay time on the basis of the propagation delay time stored in the memory.

With this configuration, by storing the measured propagation delay time once, there is no need to remeasure the propagation delay time.

The aforementioned aspects provide an advantage in that it is possible to accurately detect reflected light of the laser beam coming from an object and thus to acquire an accurate image of the object.

### REFERENCE SIGNS LIST

1 optical-scanning observation device
2 insertion part
3 laser output unit
4 illumination optical fiber
5 light-receiving optical fiber
6 light scanner
7 light detector
8 control device
9 photodetector
10 A/D converter
11 driving controller
12 image processor
13 main controller (controller)
14 memory (storage unit)
15 housing
16 display
17 measuring apparatus
18 memory
19 reading unit
L laser beam
L′ reflected light
S object

1. An optical-scanning observation device comprising:
a laser output unit that outputs a laser beam;
a light scanner that radiates the laser beam output from the laser output unit toward an object while scanning the laser beam in a direction intersecting an optical axis of the laser beam;
a light detector that detects reflected light of the laser beam reflected by the object; and
a controller that controls the laser output unit and the light detector such that the light detector detects the reflected light after a predetermined detection delay time has elapsed since the laser beam is output from the laser output unit,
wherein the controller sets the detection delay time on the basis of a propagation delay time from when the laser beam is output from the laser output unit to when the reflected light of the laser beam reaches the light detector,
the optical-scanning observation device further comprising:
an illumination optical fiber that guides the laser beam supplied from the laser output unit and emits the laser beam toward the object; and
a light-receiving optical fiber that receives the reflected light from the object and guides the received reflected light to the light detector,
wherein the controller sets the detection delay time on the basis of a time for the laser beam to propagate thorough the illumination optical fiber and a time for the reflected light to propagate through the light-receiving optical fiber.

2. An optical-scanning observation device comprising:
a laser output unit that outputs a laser beam;
a light scanner that radiates the laser beam output from the laser output unit toward an object while scanning the laser beam in a direction intersecting an optical axis of the laser beam;
a light detector that detects reflected light of the laser beam reflected by the object; and
a controller that controls the laser output unit and the light detector such that the light detector detects the reflected light after a predetermined detection delay time has elapsed since the laser beam is output from the laser output unit,
wherein the controller sets the detection delay time on the basis of a propagation delay time from when the laser beam is output from the laser output unit to when the reflected light of the laser beam reaches the light detector,
wherein the controller performs a propagation-delay-time measuring operation in which the propagation delay time is measured, and sets a time equal to the propagation delay time measured in the propagation-delay-time measuring operation as the detection delay time.

3. The optical-scanning observation device according to claim 2, wherein the controller performs the propagation-delay-time measuring operation by using the laser beam in a part of a period of time for which the light scanner performs scanning.

4. The optical-scanning observation device according to claim 2, further comprising a memory that stores the propagation delay time measured in the propagation-delay-time measuring operation, wherein the controller sets the detection delay time on the basis of the propagation delay time stored in the memory.

5. The optical-scanning observation device according to claim 3, further comprising a memory that stores the propagation delay time measured in the propagation-delay-time measuring operation, wherein the controller sets the detection delay time on the basis of the propagation delay time stored in the memory.
